(19) United States
(12) Patent Application Publication
(10) Pub. No.: US 2010/0273899 A1
(43) Pub. Date: Oct. 28, 2010

(54) INTEGRATED, HIGH-EFFICIENCY PROCESSES FOR BIOMASS CONVERSION TO SYNTHESIS GAS

(75) Inventor: John D. WINTER, Broomfield, CO (US)

Correspondence Address: Range Fuels, Inc.;Attn: Ryan O''Connor, 11101 West 120th Ave., Suite 200, Broomfield, CO, 80021, US

(73) Assignee: Range Fuels, Inc., Broomfield, CO (US)

(21) Appl. No.: 12/764,550

(22) Filed: Apr. 21, 2010

Related U.S. Application Data
(60) Provisional application No. 61/171,521, filed on Apr. 22, 2009.
(60) Provisional application No. 61/171,524, filed on Apr. 22, 2009.
(60) Provisional application No. 61/244,273, filed on Sep. 21, 2009.
(60) Provisional application No. 61/296,169, filed on Jan. 19, 2010.

(51) Int. Cl. C01B 3/38; C07C 1/04; C07C 27/00; C01B 3/02; B01J 8/18

(52) U.S. Cl. 518/703; 252/373; 423/648.1; 422/139

(57) ABSTRACT

The present invention provides several variations for converting biomass, and other carbon-containing feedstocks, into syngas. Some variations include pyrolyzing or torrefying biomass in a devolatilization unit to form a gas stream and char, and gasifying the char. Other variations include introducing biomass into a fluid-bed gasifier to generate a solid stream and a gas stream, followed by a partial-oxidation or reforming reactor to generate additional syngas from either, or both, of the solid or gas stream from the fluid-bed gasifier. Hot syngas is preferably subjected to heat recovery. The syngas produced by the disclosed methods may be used in any desired manner, such as conversion to liquid fuels (e.g., ethanol).

### FIELD OF THE INVENTION

The present invention generally relates to the field of processes and apparatus for the conversion of carbonaceous materials to synthesis gas.

### BACKGROUND OF THE INVENTION

Synthesis gas (hereinafter referred to as syngas) is a mixture of hydrogen (H₂) and carbon monoxide (CO). Syngas can be produced, in principle, from virtually any material containing carbon. Carbonaceous materials commonly include fossil resources such as natural gas, petroleum, coal, and lignite; and renewable resources such as lignocellulosic biomass and various carbon-rich waste materials. It is preferable to utilize a renewable resource to produce syngas because of the rising economic, environmental, and social costs associated with fossil resources.

Syngas is a platform intermediate in the chemical and biorefining industries and has a vast number of uses. Syngas can be converted into alkanes, olefins, oxygenates, and alcohols. These chemicals can be blended into, or used directly as, diesel fuel, gasoline, and other liquid fuels. Syngas can be converted to liquid fuels, for example, by methanol synthesis, mixed-alcohol synthesis, Fischer-Tropsch chemistry, and syngas fermentation to ethanol. Syngas can also be directly combusted to produce heat and power.

There exist a variety of conversion technologies to turn carbonaceous feedstocks into syngas. Biomass, however, poses both technical as well as economic challenges arising from operational difficulties and capital intensity. Improved processes are needed in the art to produce syngas from biomass, and other carbonaceous feedstocks, into syngas that is commercially suitable for conversion to liquid fuels and chemicals.

### SUMMARY OF THE INVENTION

The present invention addresses the aforementioned needs in the art.

In some variations, this invention provides a method of forming syngas, the method comprising:

(a) pyrolyzing or torrefying a carbon-containing feed material in a devolatilization unit to form a first gas stream and a first solid stream comprising char, wherein char is formed in an amount from about 20% to about 75% of the feed material by weight on a dry basis; and

(b) gasifying at least some of the char in a gasifier to form a second gas stream comprising syngas and a second solid stream comprising ash.

In some embodiments, the char is formed in an amount from about 20% to about 40% of the feed material by weight on a dry basis. Various embodiments may form char in an amount of about 20%, 25%, 30%, 35%, 40%, 45%, 50%, 55%, 60%, 65%, 70%, or 75% (including any intermediate percentages) of the feed material by weight on a dry basis.

Some methods further comprise thermally processing at least some of the first gas stream and/or at least some of the second gas stream. Thermally processing may include tar cracking, which is optionally performed in the presence of a catalyst suitable for tar cracking. Thermally processing may include steam reforming, which is optionally performed in the presence of a suitable reforming catalyst. Step (b) may be conducted in the presence of oxygen, steam, or both of these.

In some variations, the invention provides a method of forming syngas, the method comprising:

(a) pyrolyzing or torrefying a carbon-containing feed material in a devolatilization unit to form a first gas stream and a first solid stream comprising char, wherein char is formed in an amount from about 20% to about 75% of the feed material by weight on a dry basis; and

(b) combusting at least some of the char to produce steam, wherein a portion of the steam is recycled to step (a) to provide heat and/or to feed directly into the devolatilization unit.

In step (b), the char may be co-combusted with another solid fuel, which is not particularly limited.

Some embodiments further comprise thermally processing at least some of the first gas stream. This thermal processing may include tar cracking and/or steam reforming.

Variations of this invention provide a method of forming syngas, the method comprising:

(a) pyrolyzing or torrefying a carbon-containing feed material in a devolatilization unit to form a first gas stream and a first solid stream comprising char, wherein char is formed in an amount from about 20% to about 75% of the feed material by weight on a dry basis;

(b) gasifying at least some of the char in a gasifier to form a second gas stream comprising syngas and a second solid stream comprising ash; and

(c) feeding at least some of the first gas stream to the gasifier.

The char, in some embodiments, is formed in an amount from about 20% to about 40% of the feed material by weight on a dry basis.

In some embodiments, step (c) further includes removal of tars and/or particulates by one or more means selected from the group consisting of cooling, barrier filters, and guard beds.

At least some of the second gas stream may be subjected to thermal processing, such as tar cracking or steam reforming, either of these optionally catalyzed.

During step (c), a portion of the first gas stream may be injected at a plurality of locations within the gasifier, if desired.

Variations of the invention provide a method of forming syngas, the method comprising:

(a) pyrolyzing or torrefying biomass in a devolatilization unit to form a first gas stream and a first solid stream comprising char, wherein char is formed in an amount from about 20% to about 40% of the feed material by weight on a dry basis;

(b) gasifying the char in a gasifier to form a second gas stream comprising syngas and a second solid stream comprising ash;

(c) feeding at least some of the first gas stream to the gasifier; and

(d) thermally processing at least some of the second gas stream, wherein the thermally processing causes cracking and/or reforming reactions.

Variations of the invention provide a method of forming syngas, the method comprising:

(a) pyrolyzing or torrefying a carbon-containing feed material in a devolatilization unit to form a first gas stream and a first solid stream comprising char, wherein char is formed in an amount from about 20% to about 75% of the feed material by weight on a dry basis;

(b) gasifying at least some of the char in a gasifier to form a second gas stream comprising syngas and a second solid stream comprising ash; and

(c) thermally processing, in a reformer unit, at least some of the first gas stream and/or at least some of the second gas stream.

The thermal processing may include introducing oxygen into the reformer unit to cause some partial or total oxidation, thereby increasing the temperature during the thermal processing. The thermal processing may include catalyzed tar cracking. In some embodiments, the thermal processing is conducted on at least some of the first gas stream but not on the second gas stream. In other embodiments, the thermal processing is conducted on at least some of the second gas stream but not on the first gas stream.

Certain embodiments comprise introduction of a sub-stoichiometric amount of oxygen in a pre-combustion step prior to the thermally processing, wherein in the pre-combustion step, the oxygen causes some partial or total oxidation to increase the temperature within the reformer unit. Some of the oxygen fed to the pre-combustion step may enter the reformer unit, i.e. of the oxygen is not entirely consumed. Optionally, a supplemental fuel (which may be any fuel) is fed to the pre-combustion step.

In preferred embodiments, the thermal processing generates additional syngas which can be combined with the primary syngas stream or used in any other manner. In various embodiments, syngas may be converted to a product selected from the group consisting of alcohols, alkanes, olefins, aldehydes, ethers, acids, and hydrogen.

The present invention provides systems, process configurations, and apparatus for forming syngas, comprising:

(a) a devolatilization unit suitable for pyrolyzing or torrefying a carbon-containing feed material to form a first gas stream and a first solid stream comprising char, wherein char is formed in an amount from about 20% to about 75% of the feed material by weight on a dry basis; and

(b) a gasifier suitable for gasifying at least some of the char to form a second gas stream comprising syngas and a second solid stream comprising ash.

In some variations, the invention provides an apparatus for forming syngas, comprising:

(a) a devolatilization unit suitable for pyrolyzing or torrefying a carbon-containing feed material to form a first gas stream and a first solid stream comprising char, wherein char is formed in an amount from about 20% to about 75% of the feed material by weight on a dry basis; and

(b) a combustion device for combusting at least some of the char to produce steam, wherein a portion of the steam can be recycled to the devolatilization unit.

The invention also provides a syngas-forming apparatus comprising:

(a) a devolatilization unit suitable for pyrolyzing or torrefying a carbon-containing feed material to form a first gas stream and a first solid stream comprising char, wherein char is formed in an amount from about 20% to about 75% of the feed material by weight on a dry basis;

(b) a gasifier suitable for gasifying at least some of the char to form a second gas stream comprising syngas and a second solid stream comprising ash; and

(c) an inlet for feeding at least some of the first gas stream to the gasifier.

Some apparatus further include barrier filters or guard beds for removal of tars and/or particulates from the first gas stream, the second gas stream, or both of these.

Apparatus can also include a reactor for converting the syngas to a product selected from the group consisting of alcohols, alkanes, olefins, aldehydes, ethers, acids, and hydrogen. In some embodiments, the product includes ethanol and the apparatus includes a separation unit for recovering at least a portion of the ethanol.

Other variations and aspects of the invention are taught herein. In some variations, the present invention provides a process for converting a carbon-containing feedstock into syngas, the process comprising:

(a) introducing a carbon-containing feedstock into a fluid-bed gasifier under conditions suitable for gasifying at least a portion of the carbon-containing feedstock, thereby generating a first solid stream and a first gas stream;

(b) introducing the first solid stream and/or the first gas stream into a reactor under conditions suitable for partially oxidizing or reforming at least a portion of the first solid stream and/or the first gas stream, thereby generating a second gas stream comprising syngas at a first temperature; and

(c) introducing the second gas stream into a heat-recovery unit under suitable conditions for recovery of a portion of heat contained in the second gas stream, whereby the syngas is cooled to a second temperature lower than the first temperature.

In some embodiments, in step (b), the first solid stream is introduced into the reactor. In some embodiments, in step (b), the first gas stream is introduced into the reactor. Of course, both of the first solid stream and the first gas stream (or any portions thereof) may be introduced into the reactor.

The reactor may be an oxygen-blown reformer, or a partial-oxidation reactor (utilizing oxygen, air, or another oxidant), for example. The heat-recovery unit may be a waste-heat boiler for generating steam.

The first temperature may be selected from about 800° C. to about 1400° C., such as about 900° C. to about 1200° C. The second temperature may be less than about 400° C., such as from about 50° C. to about 350° C.

In certain variations, the invention provides a process for converting biomass into syngas, the process comprising:

(a) introducing biomass into a fluid-bed gasifier under conditions suitable for gasifying at least a portion of the biomass, thereby generating a first solid stream and a first gas stream;

(b) introducing the first solid stream and/or the first gas stream into a reactor under conditions suitable for partially oxidizing or reforming the first gas stream, thereby generating a second gas stream comprising syngas at a first temperature selected from about 800° C. to about 1200° C.;

(c) introducing the second gas stream into a heat-recovery unit under suitable conditions for recovery of a portion of heat contained in the second gas stream, whereby the syngas is cooled to a second temperature selected from about 50° C. to about 350° C.; and

(d) optionally converting at least some of the syngas into ethanol.

Some variations of the invention provide an apparatus for converting a carbon-containing feedstock into syngas, the apparatus comprising:

(a) a fluid-bed gasifier for gasifying a carbon-containing feedstock to generate a first solid stream and a first gas stream;

(b) a reactor for partially oxidizing or reforming at least a portion of the first solid stream and/or the first gas stream to generate a second gas stream comprising syngas at a first temperature; and

(c) a heat-recovery unit for recovery of a portion of heat contained in the second gas stream and for cooling the syngas to a second temperature lower than the first temperature.

The syngas produced may be used for any purpose as may be desired.

### DETAILED DESCRIPTION OF EMBODIMENTS OF THE INVENTION

This description will enable one skilled in the art to make and use the invention, and it describes several embodiments, adaptations, variations, alternatives, and uses of the invention, including what is presently believed to be the best mode of carrying out the invention.

As used in this specification and the appended claims, the singular forms “a,” “an,” and “the” include plural referents unless the context clearly indicates otherwise. Unless defined otherwise, all technical and scientific terms used herein have the same meaning as is commonly understood by one of ordinary skill in the art to which this invention belongs.

Unless otherwise indicated, all numbers expressing reaction conditions, stoichiometries, concentrations of components, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The methods and apparatus of the invention can accommodate a wide range of feedstocks of various types, sizes, and moisture contents. “Biomass,” for the purposes of the present invention, is any material not derived from fossil resources and comprising at least carbon, hydrogen, and oxygen. Biomass includes, for example, plant and plant-derived material, vegetation, agricultural waste, forestry waste, wood waste, paper waste, animal-derived waste, poultry-derived waste, and municipal solid waste. Other exemplary feedstocks include cellulose, hydrocarbons, carbohydrates or derivates thereof, and charcoal.

In various embodiments of the invention utilizing biomass, the biomass feedstock can include one or more materials selected from: timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. A person of ordinary skill in the art will readily appreciate that the feedstock options are virtually unlimited.

The present invention can also be used for carbon-containing feedstocks other than biomass, such as a fossil fuel (e.g., coal or petroleum coke), or any mixtures of biomass and fossil fuels. For the avoidance of doubt, any method, apparatus, or system described herein can be used with any carbonaceous feedstock.

Selection of a particular feedstock or feedstocks is not regarded as technically critical, but is carried out in a manner that tends to favor an economical process. Typically, regardless of the feedstocks chosen, there can be (in some embodiments) screening to remove undesirable materials. The feedstock can optionally be dried prior to processing. Optionally, particle-size reduction can be employed prior to conversion of the feedstock to syngas. Particle size is not, however, regarded as critical to the invention.

In some variations of the present invention, an improved process configuration comprises a fluid-bed gasifier followed by an oxygen-blown reformer or partial-oxidation reactor, followed by a waste-heat boiler (WHB). The addition of a WHB allows for the recovery of a portion of the exothermic heat contained in the stream exiting the reformer or partial-oxidation reactor. Generally speaking, a WHB is similar to a conventional boiler except that it is heated by means of a process gas stream instead of its own burner.

The WHB can be designed and/or operated to produce steam or hot water by heating water. The WHB can also be designed and/or operated to heat (directly or indirectly) oil, gas, or any other material. Typically, steam is produced by the WHB. This steam can be used to drive machinery directly, or to generate power via a turbo-alternator. Alternatively, or additionally, the steam can provide heat for process services, such as wood drying or alcohol distillation. Steam can also be injected directly into the fluid-bed gasifier and/or the reformer (or partial-oxidation reactor). In addition, heat available in the WHB can be used to heat other process streams, including gas streams that are fed directly, or used to heat indirectly, any unit operation within the process.

In some embodiments, a partial-oxidation or reforming reactor placed upstream of the WHB allows for substantially higher WHB inlet temperatures compared to WHB inlet temperatures in the absence of the partial-oxidation or reforming reactor. For example, WHB inlet temperatures may be increased by at least 50° C., 100° C., 200° C., 300° C. or more, when the stream exiting a fluid-bed gasifier is first send to a partial-oxidation or reforming reactor. Higher inlet temperatures to the WHB will tend to increase overall process energy efficiency.

Another advantage to carrying out reforming and/or partial oxidation ahead of the WHB is to remove tars, aromatics, and other heavy hydrocarbon species, enabling one to employ a simpler and less-expensive WHB. For example, the WHB can comprise a fire-tube boiler followed by a water-tube superheater and economizer.

There are many variations within the scheme of fluid-bed gasification followed by partial oxidation/reforming following by waste-heat boiling. A wide variety of process conditions can be employed. In addition, various gasifying, reforming, partial-oxidation, and combustion agents can be utilized throughout the process.

Furthermore, solid, liquid, and gas streams produced or existing within the process can be independently passed to subsequent steps or removed/purged from the process at any point. For example, after the fluid-bed gasification step, all or a portion of the gas can be introduced to the partial oxidizer/reformer step. Independently, all or a portion of the solid (which did not gasify or which gasified but later condensed) can be introduced to the partial oxidizer/reformer step.

In some variations, this invention provides a method of forming syngas, comprising (a) pyrolyzing or torrefying a carbon-containing feed material (such as biomass) in a devolatilization unit to form a first gas stream and a first solid stream comprising char, wherein char is formed in an amount from about 20% to about 75% of the feed material by weight on a dry basis; and (b) gasifying the char in a gasifier to form a second gas stream comprising syngas and a second solid stream comprising ash.

In some variations, this invention provides an apparatus for forming syngas, the apparatus comprising: (a) a devolatilization unit suitable for pyrolyzing or torrefying a carbon-containing feed material to form a first gas stream and a first solid stream comprising char, wherein char is formed in an amount from about 20% to about 75% of the feed material by weight on a dry basis; and (b) a gasifier suitable for gasifying at least some of the char to form a second gas stream comprising syngas and a second solid stream comprising ash.

Char is itself a carbonaceous intermediate that can be produced by pyrolysis or torrefaction. For present purposes, “char” can include liquid components, such as tars, that are not devolatilized.

“Pyrolysis” and “pyrolyze” generally refer to thermal decomposition of a carbonaceous material. In pyrolysis, less oxygen is present than is required for complete combustion of the material, such as less than 10%, 5%, 1%, 0.5%, 0.1%, or 0.01% of the oxygen that is required for complete combustion. In some embodiments, pyrolysis is performed in the absence of oxygen. The solid material remaining after pyrolysis can be referred to as char.

“Torrefaction” and “torrefy” generally refer to mild pyrolysis. Torrefaction comprises slow heating of a carbonaceous material in an inert or substantially inert atmosphere. Torrefaction reduces moisture content and produces a material (char) with a higher energy content compared to the initial material.

The pyrolysis or torrefaction step can be performed in the absence of free oxygen. In other embodiments, the pyrolysis or torrefaction step is performed in the presence of free oxygen in an amount between about 0.1% and about 25% of the stoichiometric amount of oxygen to completely combust the feed material. In some embodiments, the pyrolysis or torrefaction step is conducted in the presence of an effective catalyst.

This invention is premised on the realization, at least in part, that for economic syngas production it can be beneficial to operate the pyrolysis or torrefaction step so that char is intentionally formed in an amount greater than 15 wt % (dry basis) of the feed material. During pyrolysis or torrefaction, char can be formed in an amount from about 20% to about 75% of the feed material by weight on a dry basis. In various embodiments, the char production can be, for example, about 20%, 25%, 30%, 35%, 40%, 45%, 50%, 55%, 60%, 65%, 70%, and 75%, including all intermediate ranges. Preferred embodiments of the invention can provide for reduced overall capital costs, higher overall conversion efficiencies and energy usage, improved reliability, and/or better syngas availability.

In some variations of the invention, char is gasified to produce a gas stream that includes syngas. “Gasification” and “gasify” generally refer to the reactive generation of a mixture of at least CO, CO₂, and H₂, using oxygen and/or steam as the reactant(s). Any known gasifier can be employed. In variations, the gasifier type is entrained-flow slagging, entrained flow non-slagging, transport, bubbling fluidized bed, circulating fluidized bed, and fixed bed.

If gasification is incomplete, a solid stream can be generated, containing some of the carbon initially in the feed material. The solid stream produced from the gasification step can include ash, metals, unreacted char, and unreactive refractory tars and polymeric species. Generally speaking, feedstocks such as biomass contain non-volatile species, including silica and various metals, which are not readily released during pyrolysis, torrefaction, or gasification. It is of course possible to utilize ash-free feedstocks, in which case there should not be substantial quantities of ash in the solid stream from the gasification step.

In some embodiments, the gas stream from pyrolysis or torrefaction can be further subjected to thermal processing. In these or other embodiments, the gas stream from gasification can be subjected to thermal processing, which can be independently selected from the thermal processing of the first gas stream. Thermal processing can include steam reforming and/or partial oxidation; “steam reforming” refers to the production of syngas when steam is the reactant, while “partial oxidation” refers to the production of syngas when oxygen is the reactant. Generally, thermal processing can include one or more of devolatilization, steam reforming, partial oxidation, pyrolysis, cracking, combustion, water-gas shift, and other chemical reactions. Thermal processing can be conducted in the presence or absence of catalysts.

Thermal processing can be beneficial, for example, to convert methane to syngas, to reduce the molecular weight of tars and condensables present, to break down aromatic species, and so forth. In some embodiments, thermal processing is performed in the presence of a reforming catalyst. In some embodiments, thermal processing is performed in the presence of a catalyst suitable for tar cracking. Conditions for thermal processing can vary widely, but will include some form of heating. Heating can be internal, external, or both, and can be direct or indirect, generally being accomplished by one or mechanisms selected from convection, conduction, and thermal radiation.

In some embodiments of the invention, a portion or the entire gas stream from the pyrolysis or torrefaction step is fed to the gasification step, which can have similar or additional benefits compared to the optional thermal processing described above. When a gas stream from the devolatilization unit is fed to the gasifier, it can be preferable to remove tars and/or particulates by cooling, in conjunction with barrier filters, and guard beds, or similar means. The gas can be fed to the gasifier at any suitable location, such as the freeboard. In some embodiments, gas from the pyrolysis or torrefaction step is injected at a plurality of locations in the gasifier, to enhance desired reactions.

Other variations of the invention provide a method of forming syngas, comprising (a) pyrolyzing or torrefying a carbon-containing feed material in a devolatilization unit to form a first gas stream and a first solid stream comprising char, wherein char is formed in an amount from about 20% to about 75% of the feed material by weight on a dry basis; and (b) combusting at least some of the char to produce steam.

The char can optionally be co-combusted with another solid fuel, such as coal, charcoal, or even wood. The steam can be routed as process steam, or employed to generate electricity, or subjected to any other use. In some embodiments, a portion of the steam is recycled to the devolatilization unit to provide heat. In some embodiments, a portion of steam (from char combustion) is fed directly into the devolatilization unit.

In embodiments employing char combustion, the char production in the devolatilization unit can be, for example, about 20%, 25%, 30%, 35%, 40%, 45%, 50%, 55%, 60%, 65%, 70%, and 75%, including all intermediate ranges. The gas stream from pyrolysis or torrefaction (in the devolatilization unit) can be further subjected to thermal processing, as described above with reference to variations employing char gasification.

Exemplary changes that may occur during pyrolysis include any of the following: (i) heat transfer from a heat source increases the temperature inside the feedstock; (ii) the initiation of primary pyrolysis reactions at this higher temperature releases volatiles and forms a char; (iii) the flow of hot volatiles toward cooler solids results in heat transfer between hot volatiles and cooler unpyrolyzed feedstock; (iv) condensation of some of the volatiles in the cooler parts of the feedstock, followed by secondary reactions, can produce tar; (v) autocatalytic secondary pyrolysis reactions proceed while primary pyrolytic reactions simultaneously occur in competition; and (vi) further thermal decomposition, reforming, water-gas shift reactions, free-radical recombination, and/or dehydrations can also occur, which are a function of the residence time, temperature, and pressure profile.

Pyrolysis can at least partially dehydrate the feedstock. In various embodiments, pyrolysis removes greater than about 10%, 20%, 30%, 40%, 50%, 60%, 70%, 80%, 90% or more of the water from the feedstock. It can be beneficial, but not necessary, to remove at least 90% of the water initially present.

Any pyrolysis reactor can be used to pyrolyze the feed material. Exemplary reactor configurations include, but are not limited to, augers, ablative reactors, rotating cones, fluidized-bed reactors, entrained-flow reactors, vacuum moving-bed reactors, transport-bed reactors, fixed-bed reactors, and microwave-assisted pyrolysis reactors. In some embodiments, the devolatilization unit is provided as described in U.S. Pat. No. 6,863,878 or 7,655,215, which are each incorporated herein by reference in their entireties. In some embodiments, the devolatilization unit is provided as described in U.S. patent application Ser. No. 12/166,167 entitled “METHODS AND APPARATUS FOR PRODUCING SYNGAS” (filed Jul. 1, 2008), which is incorporated by reference herein.

In some embodiments in which an auger is used, sand or another heat carrier can optionally be employed. For example, the feedstock and sand can be fed at one end of a screw. The screw mixes the sand and feedstock and conveys them through the reactor. The screw can provide good control of the feedstock residence time and does not dilute the pyrolyzed products with a carrier or fluidizing gas. The sand can be reheated in a separate vessel.

In some embodiments in which an ablative process is used, the feedstock is moved at a high speed against a hot metal surface. Ablation of any char forming at surfaces can maintain a high rate of heat transfer. Such apparatus can prevent dilution of products. As an alternative, the feedstock particles may be suspended in a carrier gas and introduced at a high speed through a cyclone whose wall is heated.

In some embodiments in which a fluidized-bed reactor is used, the feedstock can be introduced into a bed of hot sand fluidized by a gas, which is typically a recirculated product gas. Reference herein to “sand” shall also include similar, substantially inert materials, such as glass particles, recovered ash particles, and the like. High heat-transfer rates from fluidized sand can result in rapid heating of the feedstock. There can be some ablation by attrition with the sand particles. Heat is usually provided by heat-exchanger tubes through which hot combustion gas flows.

Circulating fluidized-bed reactors can be employed, wherein gas, sand, and feedstock move together. Exemplary transport gases include recirculated product gases and combustion gases. High heat-transfer rates from the sand ensure rapid heating of the feedstock, and ablation is expected to be stronger than with regular fluidized beds. A separator can be employed to separate the product gases from the sand and char particles. The sand particles can be reheated in a fluidized burner vessel and recycled to the reactor.

In some embodiments, fast pyrolysis is used. Fast pyrolysis is a high-temperature process in which feedstock is rapidly heated. The feedstock decomposes to generate vapors and char, and (in some embodiments) a liquid phase. After cooling and condensation of the vapors, a liquid can be formed.

Pyrolysis can be performed in the presence of a catalyst. Exemplary pyrolysis catalysts include (but are by no means limited to) heterogeneous catalysts, such as SiO₂—Al₂O₃, Pt/SiO₂—Al₂O₃, WOₓ/ZrO₂, SOₓ/ZrO₂), zeolites, acid catalysts, clay catalysts, activated alumina, and Ni/Al co-precipitated catalysts. In some embodiments, a potassium, calcium, and/or lithium cation can be used to increase the selectivity and yield of char, relative to tar, during pyrolysis.

In some embodiments, the feedstock is finely ground before it is added to the pyrolysis reactor to facilitate high heating rates and fast heat transfer. In some embodiments, the reaction temperature is between about 150-600° C., such as about 200-400° C. In some embodiments, a pyrolysis catalyst allows a lower temperature to be used, such as about 250-450° C. The residence time can vary widely, depending on the selected pyrolysis temperature. For example, the residence time can be as short as 0.1 second or as long as 1 hour or more.

The pyrolysis pressure can also vary widely. Pyrolysis pressures are typically in the 10-100 psi range, but can be much higher if desired. In some embodiments, vacuum pyrolysis is employed, wherein the feedstock is heated in a vacuum to decrease the boiling point and/or avoid adverse chemical reactions. Slow or fast heating rates can be used.

In some embodiments, the char is not completely dehydrated or is allowed to pick up moisture prior to the gasification step. The water in the char can provide a source of steam for reaction in the gasifier, if desired.

Some variations of the present invention utilize the principles of torrefaction. Torrefaction can improve the properties of a carbon-containing feedstock (e.g., biomass). Torrefaction consists of a slow heating of feedstock in an inert atmosphere, typically to a maximum temperature of about 300° C. Torrefaction can provide a solid, reasonably uniform product with lower moisture content and higher energy content compared to the initial feedstock.

Torrefied material typically has the following properties: (i) hydrophobic nature (e.g., the material does not regain humidity in storage and therefore, unlike wood and charcoal, is stable with a well-defined composition); (ii) lower moisture content and higher energy content compared to the initial feedstock; (iii) formation of less smoke when burned; and (iv) higher density and similar mechanical strength compared to the initial feedstock.

In some embodiments, a torrefaction reactor is used to torrefy a portion of or the entire feedstock using standard methods, such as those described in WO 2007/078199 or in Bergman and Kiel, “Torrefaction for biomass upgrading,” 14th European Biomass Conference & Exhibition, ENC-RX-05-180, Paris, France, 2005, which publications are each hereby incorporated by reference in their entireties.

Any known torrefaction reactor can be used to torrefy the feedstock. Exemplary reactor configurations include, but are not limited to, augers, ablative reactors, rotating cones, fluidized-bed reactors, entrained-flow reactors, vacuum moving-bed reactors, transport-bed reactors, fixed-bed reactors, rotary drum kilns, and roasters.

Any reaction conditions can be used to torrefy the feedstock in the torrefaction reactor. One skilled in the art can readily select a combination of temperature, pressure, and residence time that produces a dried solid as a product of the torrefaction process.

In some embodiments, the reaction temperature is between about 150-600° C., such as about 200-400° C. A variety of pressures can be used for torrefaction, such as atmospheric pressure or greater. In some embodiments, the residence time is between about 1 minute to about 8 hours. In some embodiments, torrefaction is performed in the absence of oxygen. In some embodiments, torrefaction is performed in the presence of a catalyst. Exemplary torrefaction catalysts include (but are by no means limited to) heterogeneous catalysts, such as SiO₂—Al₂O₃, Pt/SiO₂—Al₂O₃, WOₓ/ZrO₂, SOₓ/ZrO₂), zeolites, acid catalysts, clay catalysts, activated alumina, and Ni/Al co-precipitated catalysts.

The char can be transported to another location prior to char gasification or char combustion. In some embodiments, the torrefied feedstock is crushed or densified (e.g., compressed to form pellets) using standard methods to form smaller particles that are easier to transport and/or easier to mix with other feedstocks. Char can be transported by any known means, such as by truck, train, ship, barge, tractor trailer, or any other vehicle or means of conveyance.

In some embodiments, char from the devolatilization unit is added to another solid feed material, and collectively fed to a gasifier or combuster. In some embodiments, a pyrolyzed product char is combined with a torrefied product char before they are added to a gasifier. Any standard method can be used for this solids mixing. In some embodiments, a screw is used to mix the pyrolyzed products and torrefied products. In some embodiments, a feed mixer is used, such as a vertical or horizontal mixer.

When different feedstocks are used, they can be used in any ratio and they can be introduced in the same or different locations of a devolatilization unit. Any ratio of pyrolyzed products to torrefied products can be used, such as a ratio of about 1:0.01 to about 1:100 by weight. Any ratio of char to another feedstock that has not undergone pyrolysis or torrefaction can be used, such as a ratio of about 1:0.01 to about 1:100 by weight. It will be understood that the specific selection of feedstock ratios can be influenced by many factors, including economics (feedstock prices and availability), process optimization (depending on feedstock composition profiles), utility optimization, equipment optimization, and so on.

Any known gasifier can be used convert char into syngas. Exemplary reactor configurations include, but are not limited to, fixed-bed reactors (such as countercurrent or co-current fixed-bed reactors), stationary fluidized-bed reactors, circulating fluid-bed reactors, oxygen-driven fluid-bed reactors, bubbling fluid-bed reactors, pressurized fluid-bed reactors, moving-bed gasifiers, and entrained-flow reactors (such as slagging or slag bath entrained-flow reactors).

In some embodiments in which a countercurrent fixed-bed reactor is used, the reactor consists of a fixed bed of a feedstock through which a gasification agent (such as steam, oxygen, and/or air) flows in countercurrent configuration. The ash is either removed dry or as a slag.

In some embodiments in which a cocurrent fixed-bed reactor is used, the reactor is similar to the countercurrent type, but the gasification agent gas flows in cocurrent configuration with the feedstock. Heat is added to the upper part of the bed, either by combusting small amounts of the feedstock or from external heat sources. The produced gas leaves the reactor at a high temperature, and much of this heat is transferred to the gasification agent added in the top of the bed, resulting in good energy efficiency. Since tars pass through a hot bed of char in this configuration, tar levels are expected to be much lower than when using the countercurrent type.

In some embodiments in which a fluidized-bed reactor is used, the feedstock is fluidized in oxygen and steam or air. The ash is removed dry or as heavy agglomerates that defluidize. Recycle or subsequent combustion of solids can be used to increase conversion. Fluidized-bed reactors are useful for feedstocks that form highly corrosive ash that would damage the walls of slagging reactors.

In some embodiments in which an entrained-flow reactor is used, char is gasified with oxygen or air in cocurrent flow. The gasification reactions take place in a dense cloud of very fine particles. High temperatures can be employed, thereby providing for low quantities of tar and methane in the product gas.

Entrained-flow reactors remove the major part of the ash as a slag, as the operating temperature is typically well above the ash fusion temperature. A smaller fraction of the ash is produced either as a very fine dry fly ash or as a fly-ash slurry. Some feedstocks, in particular certain types of biomass, can form slag that is corrosive. Certain entrained-bed reactors have an inner water- or steam-cooled wall covered with partially solidified slag.

Some feedstocks have ashes with very high ash-fusion temperatures. In this case, limestone can be mixed with the char prior to gasification, when slagging-type gasifiers are employed. Addition of limestone usually can lower the fusion temperature.

In some embodiments using a fluidized-bed gasifier, and without being limited by any particular theory or hypothesis, char particles can improve fluid dynamics within the gasifier. The presence of char particles with suitable properties (e.g., particle size) can reduce mass-transfer limitations and thus increase gasification rate, either prior to gasification of the char itself or in conjunction with gasification reactions.

The gas phase from the devolatilization unit can include aromatic compounds such as benzene, toluene, ethylbenzene, and xylenes (collectively known as “BTEX”). Furthermore, the gas phase from the devolatilization unit can include heavier aromatic compounds, such as refractory tars. In some embodiments, the gasifier is effective for reduction of molecular weight of refractory tars present in the input stream to the gasifier. Substantial destruction of refractory tars can produce BTEX components. In certain embodiments, the gasifier is also effective for destruction of at least a portion of BTEX species, such as conversion to carbon oxides and/or light hydrocarbons.

Some variations of the invention utilize direct-fired reforming. In some embodiments, direct-fired reforming comprises the following steps:

(a) pyrolyzing or torrefying a carbon-containing feed material in a devolatilization unit to form a first gas stream and a first solid stream comprising char, wherein char is formed in an amount from about 20% to about 75% of the feed material by weight on a dry basis;

(b) gasifying at least some of the char in a gasifier to form a second gas stream comprising syngas and a second solid stream comprising ash; and

(c) thermal processing, in a reformer unit, at least some of the first gas stream and/or at least some of the second gas stream.

In some embodiments, the thermal processing comprises introducing oxygen into the reformer unit to cause some partial or total oxidation, thereby increasing the temperature during the thermal processing. Thermal processing can include catalyzed or uncatalyzed tar cracking.

Thermal processing can be conducted on at least some of the first gas stream but not on the second gas stream. Or, thermal processing can be conducted on at least some of the second gas stream but not on the first gas stream. Of course, both of these streams could be treated in the reformer unit. Thermal processing can generate syngas when stream reforming or partial oxidation are promoted.

In some embodiments, a sub-stoichiometric amount of oxygen is introduced in a pre-combustion step prior to the thermal processing, wherein in the pre-combustion step, the oxygen causes some partial or total oxidation to increase the temperature within the reformer unit.

Some of the oxygen fed to the pre-combustion step can enter the reformer unit, if there is incomplete conversion in the pre-combustion unit. In other embodiments, none of the oxygen fed to the pre-combustion step enters the reformer unit.

Preferably, the increase in temperature within the reformer unit is effective to promote tar cracking. If desired, a supplemental fuel can be fed to the pre-combustion step. This supplemental fuel can be natural gas, waste syngas, methanol, char, or some other carbon-containing material.

The syngas produced by the methods or apparatus of the invention can be converted to one or more commercially useful products. In some variations, the syngas is filtered, purified, or otherwise conditioned prior to being converted to another product. For example, syngas may be purified wherein BTEX, sulfur compounds, nitrogen, metals, and/or other impurities are optionally removed from the syngas.

The syngas produced as described according to the present invention can generally be chemically converted and/or purified into hydrogen, carbon monoxide, methane, graphite, olefins (such as ethylene), oxygenates (such as dimethyl ether), alcohols (such as methanol and ethanol), paraffins, and other hydrocarbons. Syngas can be converted into linear or branched C₅-C₁₅ hydrocarbons, diesel fuel, gasoline, waxes, or olefins by Fischer-Tropsch chemistry; methanol, ethanol, and mixed alcohols by a variety of catalysts; isobutane by isosynthesis; ammonia by hydrogen production followed by the Haber process; aldehydes and alcohols by oxosynthesis; and many derivatives of methanol including dimethyl ether, acetic acid, ethylene, propylene, and formaldehyde by various processes.

In some embodiments, the syngas is converted to methanol using known methanol catalysts. In some embodiments, the syngas is converted to fuel components using known Fischer-Tropsch catalysts. In certain embodiments, the syngas is converted to mixed alcohols, particularly ethanol. Syngas can be selectively converted to ethanol by means of a chemical catalyst, such as described in U.S. patent application Ser. No. 12/166,203, entitled “METHODS AND APPARATUS FOR PRODUCING ALCOHOLS FROM SYNGAS,” filed Jul. 1, 2008, which is hereby incorporated herein by reference. As is known in the art, syngas can also be fermented to ethanol using various microorganisms.

The syngas produced according to the methods and apparatus of the invention can also be converted to energy. Syngas-based energy-conversion devices include a solid-oxide fuel cell, Stirling engine, micro-turbine, internal combustion engine, thermo-electric generator, scroll expander, gas burner, or thermo-photovoltaic device.

In this detailed description, reference has been made to multiple embodiments of the invention and non-limiting examples relating to how the invention can be understood and practiced. Other embodiments that do not provide all of the features and advantages set forth herein may be utilized, without departing from the spirit and scope of the present invention. This invention incorporates routine experimentation and optimization of the methods and systems described herein. Such modifications and variations are considered to be within the scope of the invention defined by the claims.

All publications, patents, and patent applications cited in this specification are herein incorporated by reference in their entirety as if each publication, patent, or patent application were specifically and individually put forth herein.

Where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially.

Therefore, to the extent there are variations of the invention, which are within the spirit of the disclosure or equivalent to the inventions found in the appended claims, it is the intent that this patent will cover those variations as well. The present invention shall only be limited by what is claimed.

What is claimed is:

1. A process for converting a carbon-containing feedstock into syngas, said process comprising:
(a) introducing a carbon-containing feedstock into a fluid-bed gasifier under conditions suitable for gasifying at least a portion of said carbon-containing feedstock, thereby generating a first solid stream and a first gas stream;
(b) introducing said first solid stream and/or said first gas stream into a reactor under conditions suitable for partially oxidizing or reforming at least a portion of said first solid stream and/or said first gas stream, thereby generating a second gas stream comprising syngas at a first temperature; and
(c) introducing said second gas stream into a heat-recovery unit under suitable conditions for recovery of a portion of heat contained in said second gas stream, whereby said syngas is cooled to a second temperature lower than said first temperature.

2. The process of claim 1, wherein in step (b), said first solid stream is introduced into said reactor.

3. The process of claim 1, wherein in step (b), said first gas stream is introduced into said reactor.

4. The process of claim 1, wherein in step (b), both of said first solid stream and said first gas stream are introduced into said reactor.

5. The process of claim 1, wherein said reactor is an oxygen-blown reformer.

6. The process of claim 1, wherein said reactor is a partial-oxidation reactor.

7. The process of claim 1, wherein said heat-recovery unit is a waste-heat boiler for generating steam.

8. The process of claim 1, wherein said first temperature is selected from about 800° C. to about 1400° C.

9. The process of claim 8, wherein said first temperature is selected from about 900° C. to about 1200° C.

10. The process of claim 1, wherein said second temperature is less than about 400° C.

11. The process of claim 10, wherein said second temperature is selected from about 50° C. to about 350° C.

12. The process of claim 1, further comprising the step of converting said syngas to a product selected from the group consisting of alcohols, alkanes, olefins, aldehydes, ethers, acids, and hydrogen.

13. The process of claim 12, wherein said product includes ethanol.

14. A process for converting biomass into ethanol, said process comprising:
(a) introducing biomass into a fluid-bed gasifier under conditions suitable for gasifying at least a portion of said biomass, thereby generating a first solid stream and a first gas stream;
(b) introducing said first solid stream and/or said first gas stream into a reactor under conditions suitable for partially oxidizing or reforming said first gas stream, thereby generating a second gas stream comprising syngas at a first temperature selected from about 800° C. to about 1200° C.;
(c) introducing said second gas stream into a heat-recovery unit under suitable conditions for recovery of a portion of heat contained in said second gas stream, whereby said syngas is cooled to a second temperature selected from about 50° C. to about 350° C.; and
(d) converting said syngas into a product comprising ethanol.

15. An apparatus for converting a carbon-containing feedstock into syngas, said apparatus comprising:
(a) a fluid-bed gasifier for gasifying a carbon-containing feedstock to generate a first solid stream and a first gas stream;
(b) a reactor for partially oxidizing or reforming at least a portion of said first solid stream and/or said first gas stream to generate a second gas stream comprising syngas at a first temperature; and
(c) a heat-recovery unit for recovery of a portion of heat contained in said second gas stream and for cooling said syngas to a second temperature lower than said first temperature.

16. The apparatus of claim 15, wherein said reactor is an oxygen-blown reformer.

17. The apparatus of claim 15, wherein said reactor is a partial-oxidation reactor.

18. The apparatus of claim 15, wherein said heat-recovery unit is a waste-heat boiler for generating steam.

19. The apparatus of claim 15, further comprising a unit for converting said syngas to a product selected from the group consisting of alcohols, alkanes, olefins, aldehydes, ethers, acids, and hydrogen.

20. The apparatus of claim 19, wherein said product includes ethanol.
